(19) United States
(12) Patent Application Publication
(10) Pub. No.: US 2018/0036157 A1
(43) Pub. Date: Feb. 8, 2018

(54) STENT DELIVERY SYSTEM

(71) Applicant: BOSTON SCIENTIFIC SCIMED, INC., Maple Grove, MN (US)

(72) Inventors: Matthew Montague, Oranmore (IE); Martyn G. Folan, Loughrea (IE); Thomas M. Keating, Tuam (IE); Geraldine A. Toner, Raphoe (IE)

(21) Appl. No.: 15/666,011

(22) Filed: Aug. 1, 2017

Related U.S. Application Data
(60) Provisional application No. 62/369,924, filed on Aug. 2, 2016.

(51) Int. Cl. A61F 2/966

(52) U.S. Cl. CPC A61F 2/966; A61F 2250/0013; A61M 25/09

(57) ABSTRACT

An example stent delivery system is disclosed. The example stent delivery system includes an inner shaft having a proximal portion, a distal portion and at least one lumen extending therein. The inner shaft further includes a stent receiving region disposed along the distal portion. The stent delivery system further includes a stent disposed along the stent receiving region. Additionally, the inner member includes a plurality of apertures disposed along at least a portion of the stent receiving region and the plurality of apertures are configured to permit fluid to flow therethrough and expand the stent.

### CROSS REFERENCE TO RELATED APPLICATIONS

This application claims the benefit of provisional U.S. Patent Application No. 62/369,924, filed on Aug. 2, 2016, which is hereby incorporated by reference in its entirety.

### TECHNICAL FIELD

The present disclosure pertains to medical devices and methods for making and using medical devices. More particularly, the present disclosure pertains to stent delivery systems.

### BACKGROUND

A wide variety of intracorporeal medical devices have been developed for medical use, for example, intravascular use. Some of these devices include stent delivery systems. In some instances, medical devices (e.g., self-expanding stents) are placed in the esophagus for the treatment of esophageal strictures. In other instances, an implantable medical device may be used to treat a stenosis in a blood vessel, used to maintain a fluid opening or pathway in the vascular, urinary, biliary, tracheobronchial, esophageal, or renal tracts, or position a device such as an artificial valve or filter within a body lumen, in some instances.

Stents are generally tubular shaped devices which function to expand within a segment of a body lumen, such as an esophagus, a trachea, a colon, a blood vessel, or other body lumen or cavity. Stents are usually delivered in a compressed condition to a target site and then deployed at that location into an expanded condition to support the body lumen. Self-expanding stents are generally compressed, or otherwise radially constrained to a reduced diameter during delivery that is smaller than the eventual deployed diameter at the desired site. When positioned at the desired site within the body lumen, the stent may be deployed by removing the constraining force, thereby being allowed to self-expand into the desired diameter.

In some instances, a self-expanding stent may include a covering or coating positioned on an outer surface thereof. Further, in some instances the covering or coating of a self-expanding stent may cause one or more folds of the covering or coating to self-adhere to adjacent folds (e.g., may self-adhere while the stent is folded upon itself in a constrained configuration). Therefore, a self-expanding stent may have difficulty expanding to a fully expanded diameter under its own expansion forces. Accordingly, it may be desirable to provide stent delivery systems designed to aid a self-expanding stent in deploying from a partially expanded state to a fully expanded state.

### BRIEF SUMMARY

This disclosure provides design, material, manufacturing method, and use alternatives for medical devices.

An example stent delivery system includes an inner shaft having a proximal portion, a distal portion and at least one lumen extending therein. The inner shaft further includes a stent receiving region disposed along the distal portion. The stent delivery system further includes a stent disposed along the stent receiving region. Additionally, the inner member includes a plurality of apertures disposed along at least a portion of the stent receiving region and the plurality of apertures are configured to permit fluid to flow therethrough against an inner surface of the stent to assist expansion of the stent.

Alternatively or additionally to any of the embodiments above, the stent delivery system includes a deployment sheath disposed about the inner shaft.

Alternatively or additionally to any of the embodiments above, the stent is disposed between the deployment sheath and the inner shaft.

Alternatively or additionally to any of the embodiments above, each of the plurality of apertures is in fluid communication with the at least one lumen of the inner shaft.

Alternatively or additionally to any of the embodiments above, the at least one lumen extending within the inner shaft is configured to permit a guidewire to extend therethrough.

Alternatively or additionally to any of the embodiments above, the inner shaft includes a plurality of lumens extending therein, and at least one of the plurality of lumens is in fluid communication with each of the plurality of apertures.

Alternatively or additionally to any of the embodiments above, each of the plurality of apertures are spaced apart from each other along the stent receiving region.

Alternatively or additionally to any of the embodiments above, a seal is disposed in the at least one lumen distal of the plurality of apertures.

Alternatively or additionally to any of the embodiments above, the plurality of apertures are configured to deliver fluid toward the inner surface of the stent such that the fluid expands the stent from a first position in which the stent is partially deployed to a second position in which the stent is fully deployed.

Alternatively or additionally to any of the embodiments above, the plurality of apertures are further configured to withdraw fluid therethrough in order to create a vacuum sufficient to radially collapse the stent radially inward after being expanded.

Another stent delivery system includes an inner shaft having a proximal portion, a distal portion and at least one lumen extending therein, a deployment sheath positioned over the inner shaft and a stent positioned between the inner shaft and the deployment sheath. The inner member further includes a plurality of openings disposed along the distal portion and the plurality of openings are configured to permit fluid to flow therethrough to expand the stent from a first partially deployed position to a second fully deployed position.

Alternatively or additionally to any of the embodiments above, the inner member further includes a stent receiving region positioned along the distal portion of the inner member.

Alternatively or additionally to any of the embodiments above, the plurality of openings are located along the stent receiving region.

Alternatively or additionally to any of the embodiments above, the stent is positioned along the stent receiving region such that the plurality of openings are directed at an inner surface of the stent.

Alternatively or additionally to any of the embodiments above, the plurality of openings are in fluid communication with the at least one lumen.

Alternatively or additionally to any of the embodiments above, each of the plurality of openings are spaced apart from each other along the stent receiving region.

Alternatively or additionally to any of the embodiments above, each of the plurality of openings are designed to channel fluid radially away from a longitudinal axis of the inner member.

Alternatively or additionally to any of the embodiments above, the plurality of openings are further configured to withdraw fluid therethrough, wherein the withdrawn fluid creates a vacuum such that the stent is pulled radially inward after being expanded.

Alternatively or additionally to any of the embodiments above, the inner shaft includes a plurality of lumens extending therein, and wherein at least one of the plurality of lumens is in fluid communication with each of the plurality of openings.

An example method for deploying a stent includes advancing a stent delivery system to a target site within a patient. The stent delivery system includes an inner shaft having a proximal portion, a distal portion and at least one lumen extending therein. The inner shaft further includes a stent receiving region disposed along the distal portion. The stent delivery system further includes a stent disposed along the stent receiving region. Further, the inner member includes a plurality of apertures disposed along at least a portion of the stent receiving region. The plurality of apertures are configured to permit fluid to flow therethrough. The method further comprises expelling fluid through the plurality of apertures such that the fluid expands the stent from a first partially deployed position to a second fully deployed position.

The above summary of some embodiments is not intended to describe each disclosed embodiment or every implementation of the present disclosure. The Figures, and Detailed Description, which follow, more particularly exemplify these embodiments.

### BRIEF DESCRIPTION OF DRAWINGS

The disclosure may be more completely understood in consideration of the following detailed description of various embodiments of the disclosure in connection with the accompanying drawings, in which:

FIG. 1 is a cross-sectional side view of an example stent delivery system;

FIG. 2 is a cross-sectional side view of a portion of the example stent delivery system shown in FIG. 1 during a stage of deployment of a stent;

FIG. 3 is a cross-sectional view along line 3-3 of the stent delivery device shown in FIG. 2;

FIG. 4 is a cross-sectional view of the example stent delivery system during another stage of deployment of a stent;

FIG. 5 is a cross-sectional view of the example stent delivery system during another stage of deployment of a stent;

FIG. 6 is a cross-sectional view along line 6-6 of the stent delivery device shown in FIG. 5;

FIG. 7 is a cross-sectional view of another example stent delivery system;

FIG. 8 is an alternative cross-sectional view of the example stent delivery system of FIG. 7;

FIGS. 9-12 are cross-sectional views illustrating stages of retrieving a stent using the example stent delivery system of FIG. 1;

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the disclosure.

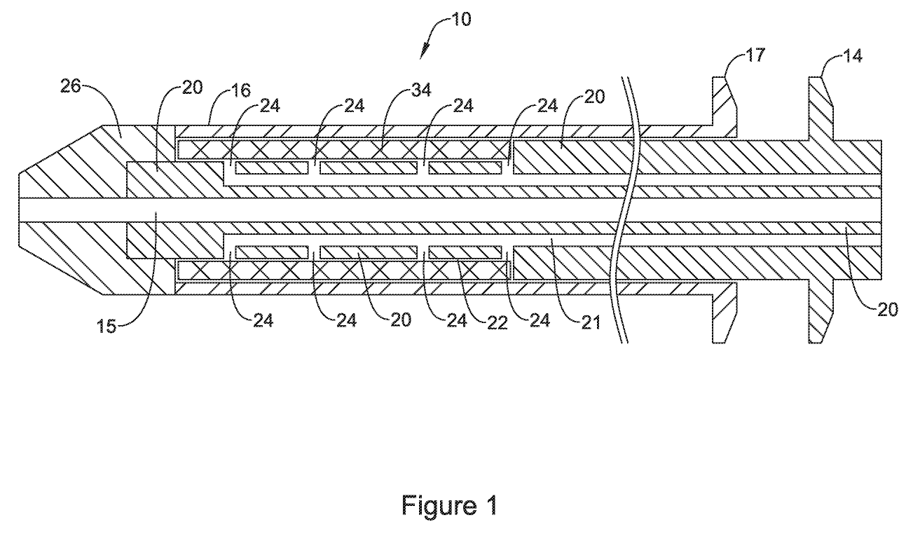

FIG. 1 illustrates an example stent delivery system 10. In general, system 10 may be configured to position a stent 34 in a body lumen for a variety of medical applications. For example, implantable medical device 10 may be used to treat a stenosis in a blood vessel, used to maintain a fluid opening or pathway in the vascular, urinary, biliary, tracheobronchial, esophageal, or renal tracts, or position a device such as an artificial valve or filter within a body lumen, in some instances. In some instances, implantable medical device 10 may include a prosthetic graft, a stent-graft, or a stent (e.g., a vascular stent, tracheal stent, bronchial stent, esophageal stent, etc.), an aortic valve, filter, etc. Although illustrated as a stent, implantable medical device 34 may be any of a number of devices that may be introduced endoscopically, subcutaneously, percutaneously or surgically to be positioned within an organ, tissue, or lumen, such as a heart, artery, vein, urethra, esophagus, trachea, bronchus, bile duct, or the like.

As stated above, a self-expanding stent may include a covering or coating positioned on an outer surface thereof. In some instances, the stent covering or coating may be include a material that is prone to adhering to itself when folded and pressed together. It can be appreciated that when a stent is radially contrained prior to being or while positioned in a stent delivery device, the folds of a stent, including the covering or coating, may come into contact with one another. It can further be appreciated that in some instances the covering or coating of a self-expanding stent may cause one or more folds on the stent to self-adhere to other, adjacent folds (e.g., the stent covering or coating may self-adhere to adjacent stent folds while the stent is folded upon itself in a constrained configuration).

Additionally, the adhesion of adjacent folds in a stent may increase the radial expansion force necessary to fully deploy the stent. For example, as the stent begins to self-expand from a first constrained configuration to a fully deployed configuration, the stent must overcome the resistance to expansion provided by adjacent folds sticking to one another. Therefore, it can be appreciated that a self-expanding stent may have difficulty expanding to a fully expanded diameter under its own expansion forces. Accordingly, it may be desirable to design stent delivery systems which overcome the adhesion forces between folds of the covering or coating, and thus aid a self-expanding stent in deploying from a partially expanded state to a fully expanded state. Examples disclosed herein provide an apparatus and methodology to provide self-expanding stents with additional radial expansion forces sufficient to overcome a stent's resistance to expansion due to adherence of adjacent stent folds.

Deployment of the stent 34 may include uncovering the stent 34 from an outer sheath disposed over the stent 34 during delivery. For example, a retraction sheath 16, which may overlie the stent 34, may be proximally retracted to uncover the stent 34 in a distal-to-proximal direction. In other instances, distal advancement of an outer sheath overlying the stent 34 may uncover the stent 34 in a proximal-to-distal direction. Longitudinal actuation (proximal retraction and/or distal advancement) of sheath 16 may include the actuation (e.g., proximal retraction and/or distal advancement) of a handle member 17 which may be coupled to the proximal end of retraction sheath 16.

As discussed above, in at least some examples disclosed herein, stent 34 may be a self-expanding stent. Self-expanding stent examples may include stents having one or more struts combined to form a rigid and/or semi-rigid stent structure. For example, stent struts may be wires or filaments braided, intertwined, interwoven, weaved, knitted, crocheted or the like to form the stent structure. The struts (e.g., wires or filaments) of the stent 34 may be configured to self-expand to an expanded diameter when unconstrained. Alternatively, stent 34 may be a monolithic structure formed from a cylindrical tubular member, such as a single, cylindrical tubular laser-cut Nitinol tubular member, in which the remaining portions of the tubular member form the stent struts. The monolithic structure of the stent 34 (e.g., struts) may be configured to self-expand to an expanded diameter when unconstrained.

Stent 34 in examples disclosed herein may be constructed from a variety of materials. For example, stent 34 may be constructed from a metal (e.g., Nitinol). In other instances, stent 34 may be constructed from a polymeric material (e.g., PET). In yet other instances, stent 34 may be constructed from a combination of metallic and polymeric materials. Additionally, stent 34 may include a bioabsorbable and/or biodegradable material.

In some instances, example stent 34 may include one or more layers (e.g., covering, coating, etc.) of material positioned on and/or adjacent to the outer surface of stent 34. In some instances, the outer layer or covering may be an elastomeric or non-elastomeric material. For example, the outer layer or covering may be a polymeric material, such as silicone, polyurethane, or the like. Further, the outer layer may span the spaces (e.g., openings, cells, interstices) between struts or filaments of stent 34.

In other examples disclosed herein, stent delivery system may 10 may include a flexible polymeric sheath which has no underlying stent support. For example, stent delivery system 10 may include a sheath or film (e.g., cylindrical foam) that may be expanded similar to how stent 34 may be expanded. For example, the sheath or film may not have any underlying stent support, yet may function and integrate with stent delivery system 10 in a similar manner as stent 34. These types of structures may be beneficial to treat bariatric leaks.

FIGS. 1-6 illustrate an example stent delivery system 10 and sequence of steps showing the deployment of stent 34 from stent delivery system 10. As illustrated in FIG. 1, system 10 may include an inner shaft or member 20. In at least some embodiments, inner member 20 may be a tubular structure and, thus, may include one or more lumens 15 and/or 21 and a handle member 14 positioned along a proximal portion thereof. For example, FIG. 1 illustrates inner member 20 including a guidewire lumen 15 that extends along at least a portion of the length of inner member 20. Accordingly, system 10 may be advanced over a guidewire to the desired target location in the body lumen. In addition, or in alternative examples, the guidewire lumen 15 may be a infusion/perfusion/aspiration lumen that allows portions, components, or all of system 10 to be flushed, perfused, aspirated, or the like.

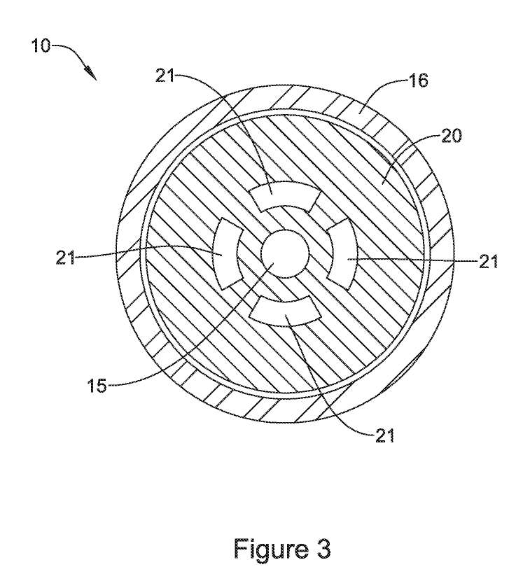

In other examples, inner member 20 may include more than one lumen. For example, inner member 20 may include one or more fluid delivery lumens 21 extending along at least a portion of the length of inner member 20. FIG. 1 illustrates one or more fluid delivery lumens 21 extending substantially parallel to and alongside guidewire lumen 15. As will be further discussed with respect to FIG. 3, a plurality of fluid delivery lumens 21 may be spaced away from one another within inner member 20.

Inner member 20 may include a stent receiving region 22 about which a stent 34 may be disposed. The length and/or configuration of stent receiving region 22 may vary. For example, stent receiving region 22 may have a length sufficient for the stent 34 to be disposed thereon in a radially compressed, constrained configuration within outer sheath 16. It can be appreciated that the length of stent 34 utilized for system 10 may dictate the desired length of stent receiving region 22 to accommodate stent 34. The guidewire lumen 15 may extend centrally through stent receiving region 22 to a distalmost extent of device 10.

Along or otherwise disposed adjacent to stent receiving region 22 may be one or more fluid delivery ports 24. Ports 24 may extend through the wall of inner member 20 such that fluid may be delivered through the lumen(s) 21 of inner member 20 and directed (e.g., channeled, funneled, etc.) through ports 24. In other words, in some examples fluid delivery lumen(s) 21 may be in fluid communication with one or more fluid delivery ports 24. Further, lumen(s) 21 may extend through inner member 20 and to a proximal port at a proximal end thereof. A source of fluid (not shown) may be coupled to the proximal port to provide fluid through the lumen(s) 21 to the ports 24. In some examples, a valve (e.g., stopcock, luer fitting, etc.) may be provided at the proximal port, or another location, such that a clinician may selectively control delivery of fluid therethrough.

As will be described in greater detail below, in some examples it may be desirable to direct fluid through ports 24 to assist radial expansion of stent 34 during deployment from system 10. For example, ports 24 may allow a clinician to expand stent 34 from a partially deployed configuration, having a first diameter, to a fully deployed configuration, having a second diameter greater than the first diameter. Additionally, in some examples, ports 24 may allow a clinician to radially contract (e.g., pull radially inward) stent 34 to reposition and/or retrieve stent 34. For example, ports 24 may allow a clinician to pull a fully deployed stent 34 radially inward toward inner member 20 such that the clinician can reposition stent 34 within the body lumen and/or recapture stent 34 within stent delivery system 10.

A tip 26 may be attached to, incorporated with, or otherwise disposed at the distal end of inner member 20. Tip 26 may generally have a tapered, rounded or smooth shape that provides a generally atraumatic distal end to system 10. For example, tip 26 may have a smooth distal portion that gently tapers in a proximal to distal direction. As illustrated in FIG. 1, in some examples a portion of inner member 20 may extend into a portion of tip 26 and be secured thereto. However, in other examples it is contemplated that tip 26 and inner member 20 may form a monolithic structure.

As stated above, FIG. 1 illustrates inner member 20 and a stent 34 disposed about inner member 20 (e.g., about stent receiving region 22 of inner member 20). In some embodiments, stent 34 is a self-expanding stent. Accordingly, stent 34 may be biased to outwardly expand. For example, stent 34 may be loaded onto inner member 20 by radially contracting stent 34 around inner member 20 in a contracted configuration. Stent 34 may then be restrained within deployment sheath 16 in the radially contracted configuration. In alternative embodiments, however, stent 34 may be directly loaded onto inner member 20 via crimping or any other suitable mechanical holding mechanism.

In at least some examples, it can be appreciated that sheath 16 is configured to shift between a first position, for example as shown in FIG. 1, where sheath 16 overlies stent 34 and a second position where stent 34 is uncovered from sheath 16, such as when sheath 16 is proximally retracted to a position substantially proximal of stent 34. In general, the first position may be utilized during navigation and delivery of system 10 to the appropriate location within a body lumen and the second position may be used to deploy stent 34.

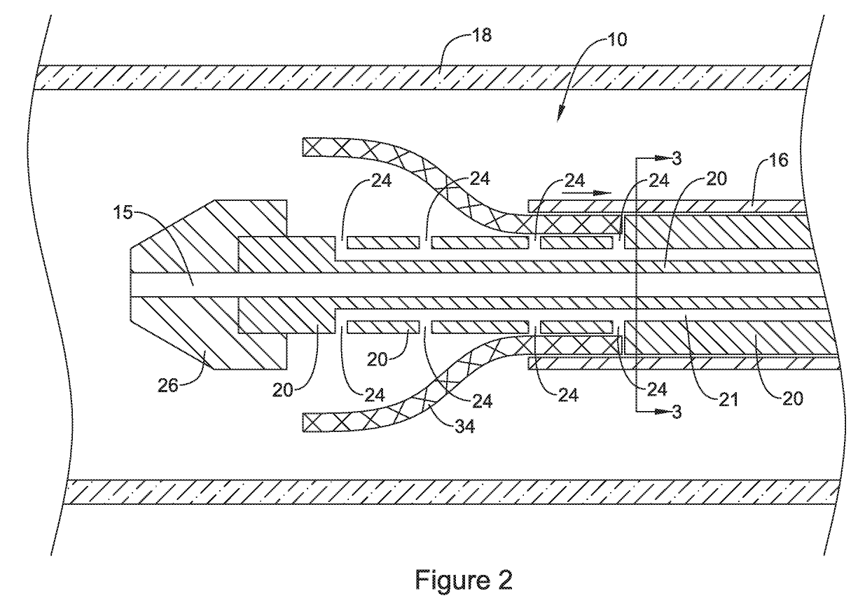

FIG. 2 illustrates an example step in the deployment of stent 34 within an example body lumen 18. It can be appreciated that prior to the deployment step illustrated in FIG. 2, system 10 may have been navigated to a position adjacent a target site in a body lumen. Once navigated to a desired location, a clinician or other operator may retract sheath 16 relative to inner member 20 and stent member 34. As stated above, stent member 34 may be a self-expanding stent biased to expand radially outward when unconstrained. Therefore, it can be appreciated that as sheath 16 is retracted in a proximal direction (thereby exposing a portion of stent 34), the exposed portion of stent 34 may automatically radially expand outward. FIG. 2 illustrates an exposed portion of stent 34 expanding radially away from inner member 20 as retraction sheath 16 is translated in a proximal direction (as depicted by the proximal pointing arrow in FIG. 2).

FIG. 3 illustrates a cross-section along line 3-3 of FIG. 2. It can be appreciated from FIG. 3 that the inner member 20 shown in the example system 10 of FIG. 1 may include a plurality of fluid delivery lumens 21. Fluid delivery lumens 21 may be spaced circumferentially away from one another around the longitudinal axis of inner member 20 and symmetrically or asymmetrically around guidewire lumen 15. For example, the cross-section of FIG. 3 shows four separate fluid delivery lumens 21. Each delivery lumen 21 may be in fluid communication with one or more, or a plurality of ports 24 positioned under stent 24 in stent receiving region 22. While FIG. 3 shows four fluid delivery lumens 21, it can be appreciated that inner member 20 may include more or less than four fluid delivery lumens 21 spaced around a central region of inner member 20. For example, inner member 20 may include 1, 2, 3, 4, 5, 6, 7, 8, 9, 10 or more fluid delivery lumens 21. Additionally, FIG. 3 shows a guidewire lumen 15 positioned in a central region of inner member 20.

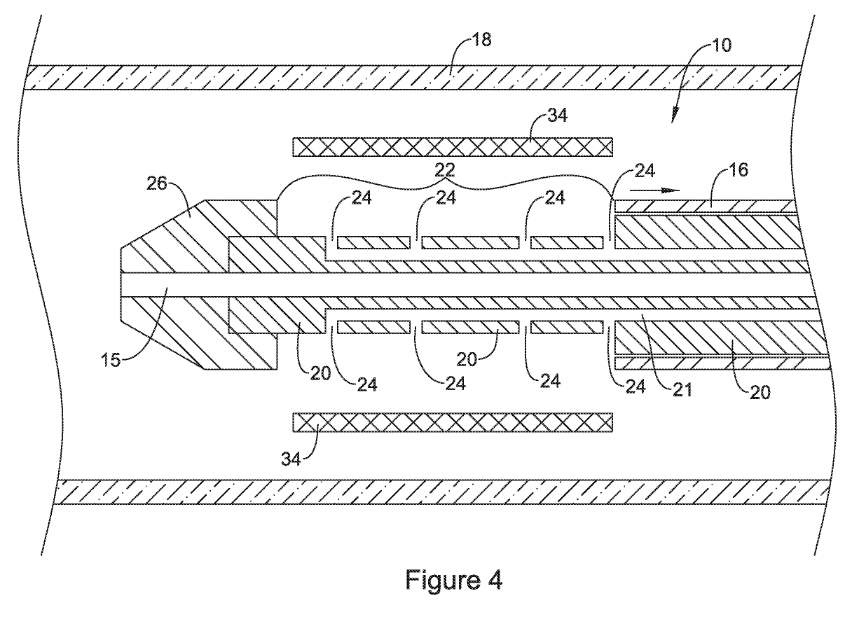

FIG. 4 illustrates an example second step in the deployment of example stent 34. As shown in FIG. 4, retraction sheath 16 may be retracted in a proximal direction such that the distal end of retraction sheath 16 is positioned proximal to the stent retaining region 22. Further, it can be appreciated that as sheath 16 is retracted proximally, stent 34 may continue to expand radially outward, thereby completely uncovering (and releasing) stent 34 from retraction sheath 16.

FIG. 4 further illustrates stent 34 having been partially deployed in example body lumen 18. As stated above, in some examples stent 34 may include a covering and/or coating (or some other structure) that restricts the outward expansion of stent 34 in the radial direction. For example, a covering of stent 34 may prevent the stent 34 from fully expanding against an inner surface of the body lumen when unconstrained by the sheath 16. In those instances, therefore, it may be desirable to design a stent delivery system 10 that can further assist stent 34 in moving from a partially deployed configuration to a fully deployed configuration in which the stent 34 is expanded against the luminal wall of the body lumen 18.

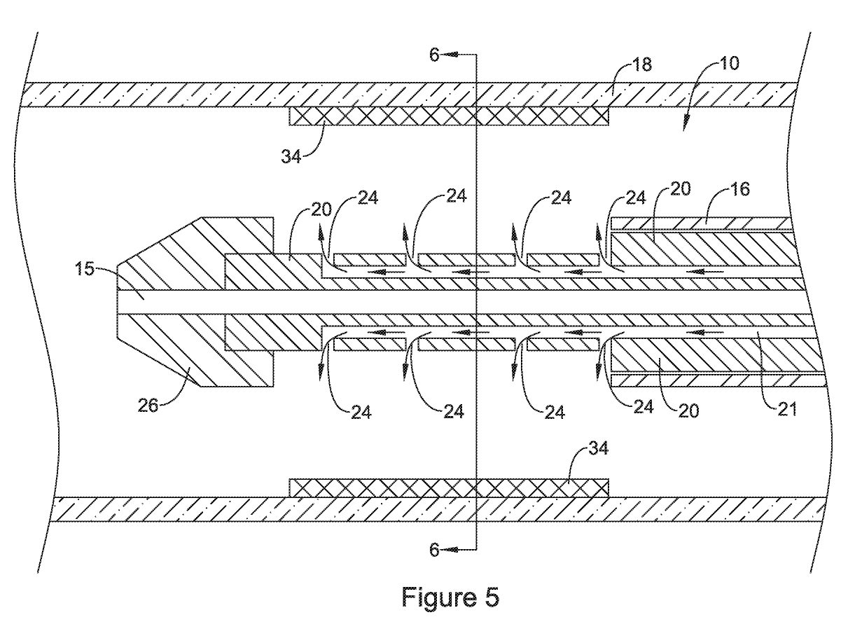

For example, FIG. 5 illustrates stent system 10 assisting stent 34 in moving from a partially deployed configuration (shown in FIG. 4) to a fully deployed configuration. As shown in FIG. 5, in at least some examples, a fluid may be directed (e.g., infused, injected, etc.) down one or more fluid delivery lumens 21 (as depicted by the arrows flowing along fluid delivery lumens 21 in FIG. 5). It should be noted that for purposes of this disclose, it is understood that the term fluid includes all liquids, gases and combinations thereof. For example, fluids may include gases or liquids, such as saline solutions, DI water, contrast agents, etc.

As further illustrated in FIG. 5, the fluid may flow through fluid delivery lumens 21 and be channeled (e.g., funneled, directed, concentrated, etc.) out of one or more fluid ports 24. As discussed above, the fluid ports 24 may be positioned along stent receiving region 22, and therefore, may be positioned such that they direct fluid toward an inner surface of example stent 34. Further, it can be appreciated that fluid directed toward the inner surface of stent 34 may assist the stent 34 in being further radially deployed or expanded into contact with the inner surface of body lumen 18. For example, ports 24 may be designed such that they channel fluid toward the inner surface of stent 34 at a sufficient flowrate, pressure, velocity, etc. to apply a radially outwardly directed force against the inner surface of stent 34 to expand the stent radially outward from a partially deployed position (as shown in FIG. 4) to a fully deployed position (as shown in FIG. 5). For example, in some instances system 10 (including fluid delivery lumens 21 and ports 24) may be designed to direct fluid out of ports 24 (toward the inner surface of stent 34) at pressures of about 1 ATM or more, about 2 ATM or more, about 5 ATM or more, about 10 ATM or more, or about 20 ATM or more. In some instance, the pressure may be in the range of about 1-20 ATM, about 5-15 ATM, or about 8-12 ATM if desired.

It can further be appreciated that the shape and or arrangement of fluid ports 24 along inner member 20 may take a variety of shapes and/or configurations. For example, fluid ports 24 may define a variety of shapes and/or orientations depending on the specific requirements necessary to aid in the expansion of stent 34. For example, ports 24 may be shaped as a nozzle and/or funnel. The nozzle/funnel shape of ports 24 may increase the fluid velocity and/or pressure of the fluid as it exits ports 24. It can be appreciated that as fluid flows distally through lumens 21, the shape of ports 24 may both re-direct the flow of fluid through ports 24 and adjust the speed and/or pressure of the fluid exiting ports 24 (which then, in turn, contacts the inner surface of stent 34).

Further, in some instances ports 24 may be directed toward specific parts or regions of stent 34 to aid in expansion according to specific design requirements. For example, ports 24 may be directed in a proximal direction, a distal direction, or in any direction (e.g., radially, longitudinally, etc.) or in any combination of directions.

Additionally, fluid ports 24 may be spaced substantially equidistant from one another along stent receiving region 22. In other examples, however, ports 24 may be spaced at unequal distances from one another along stent receiving region 22. For example, ports 24 may be arranged linearly in rows extending longitudinally along stent receiving region 22. In other examples, ports 24 may be arranged in rows offset circumferentially and/or longitudinally from one another, creating a grid-like pattern. Further, the ports 24 may be arranged in a pattern in which a subset of ports include a higher concentration of ports 24 (vs. an adjacent region including ports 24) over a given surface along stent receiving region 22. Further, some examples may include ports 24 which are arranged in helical patterns or rows along stent receiving region 22. In yet other examples, ports 24 may be arranged such that more ports 24 are positioned adjacent to the ends regions of stent 34 than in the middle portion of stent 34. Additionally, ports 24 may also be positioned such that a greater concentration of ports are located in the middle region of stent 34 versus the end regions of stent 34. In some examples, ports 24 may be aligned as radial rings being positioned around the circumference of stent receiving region 22.

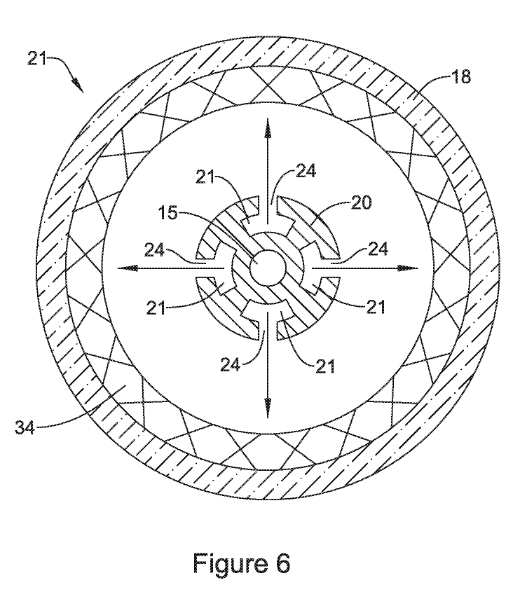

FIG. 6 illustrates a cross-section of system 10 along line 6-6 of FIG. 5. As shown by the arrows in FIG. 6, fluid may be displaced outward through ports 24 (shown in FIG. 5) via the fluid delivery lumens 21 (shown in FIG. 6). Further, as discussed above, the fluid may expand stent 34 radially outward to a position in which stent 34 is deployed against the inner surface of lumen 18.

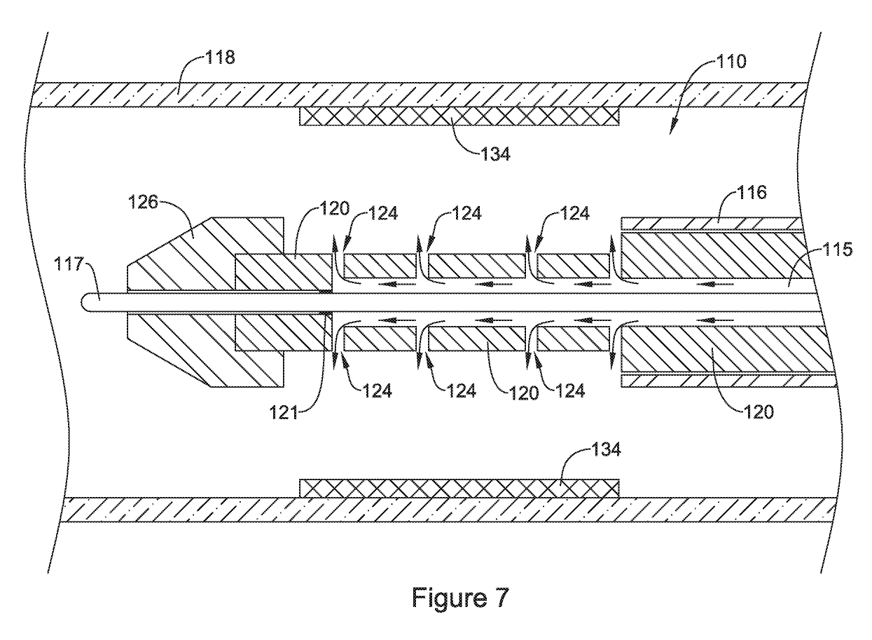

FIG. 7 illustrates another example stent delivery system 110 positioned in example body lumen 118. System 110 may operate similarly to system 10 described above with respect to FIGS. 1-6. For example, stent system 110 may include inner member 120 having ports 124, tip 126 and retraction sheath 116. However, FIG. 7 illustrates that system 110 may deliver fluid to ports 124 through guidewire lumen 115, instead of one or more discrete fluid delivery lumens. Thus, the inner member 120 may include a plurality of ports 124 in fluid communication with guidewire lumen 115. FIG. 7 shows a guidewire 117 positioned within guidewire lumen 115 and a seal 121 positioned along the inner surface of guidewire lumen 115 distal of ports 124 to form a fluid tight seal against the guidewire 117. Seal 121 may be positioned along the distal portion of inner member 120 distal of ports 124.

As shown in FIG. 7, seal 121 may surround a portion of guidewire 117 to form a seal therewith. Furthermore, seal 121 may be designed such that it prevents fluid from flowing therethrough distally of seal 121. Additionally, FIG. 7 shows fluid flowing through the annular space present between the outer surface of guidewire 117 and the inner surface of inner member 120. Further, because seal 121 is positioned distal to ports 124 (which may be similar in structure to ports 24 described above), fluid flowing in a distal direction through guidewire lumen 115 may be directed out of ports 124 in a similar manner (e.g., similar pressure, flowrate, velocity, etc.) as described above with respect to FIGS. 1-6. In other words, seal 121 may radially constrict (down on the outer surface of guidewire 117) with sufficient force such that fluid cannot flow past seal 121, and therefore, must exit fluid ports 124.

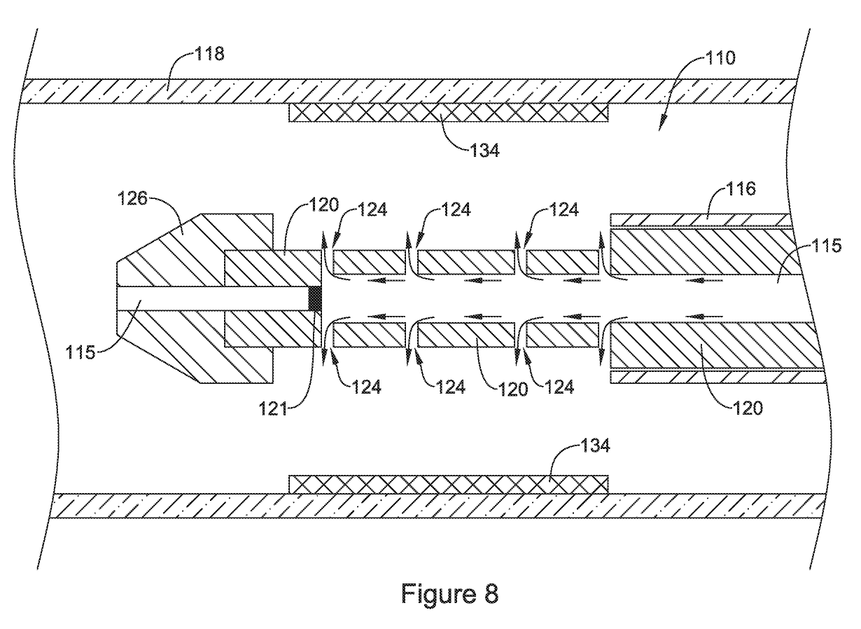

FIG. 8 illustrates an alternative configuration of stent system 110 without guidewire 117 extending through guidewire lumen 115. As can be appreciated from FIG. 8, seal 121 may be configured to radially contract and completely close off guidewire lumen 115 such that seal 121 provides a complete barrier to any fluid flowing therethrough. Therefore, it can be appreciated that system 110 may be utilized in a configuration where guidewire 117 is not positioned within guidewire lumen 115. Further, fluid may flow through guidewire lumen 115 (as depicted by the arrows in FIG. 8) and guidewire ports 124 in a manner as described in any of the examples above.

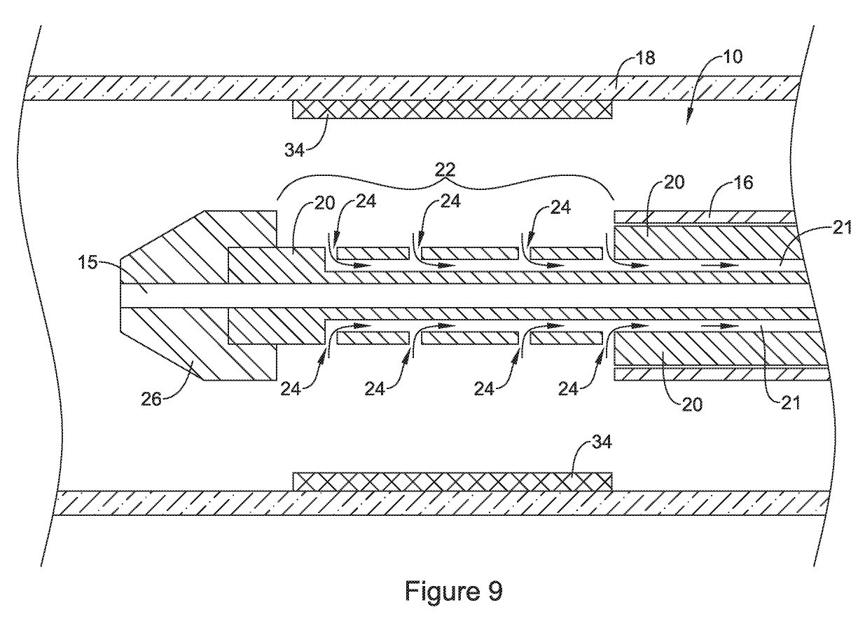

FIGS. 9-12 illustrate the example stent delivery system 10 (as described with respect to FIGS. 1-6 above) being utilized to reposition, recapture and/or remove stent 34 from inner surface of example body lumen 18.

FIG. 9 shows stent 34 positioned along the inner surface of body lumen 18. Additionally, FIG. 9 shows stent system 10 positioned such that stent receiving region 22 is positioned (e.g., axially aligned) with distal and proximal end regions of stent 34. For example, stent receiving region 22 may be positioned such that ports 24 are aligned radially inward of the inner surface of stent 34, between the ends of stent 34. Further, in some examples it is contemplated that a negative pressure (e.g., a vacuum) may be applied to the fluid delivery lumens 21. Therefore, it can further be appreciated that applying a vacuum to fluid delivery lumens 21 may create a force within example lumen 18 that pulls the inner surface of stent 34 radially inward toward stent receiving region 22 of inner member 20. In other words, the arrows shown in FIG. 9 depict the flow of fluid extending radially inward from the inner surface of stent 34, through ports 24 and proximally through fluid delivery lumens 21. In some examples, the force created by the distal-to-proximal flow of fluid through ports 24 and lumens 21 may be sufficient to “pull” stent 34 away from the inner surface of lumen 18 (e.g., radially collapse) and radially inward toward and/or against stent receiving region 22 of inner member 20.

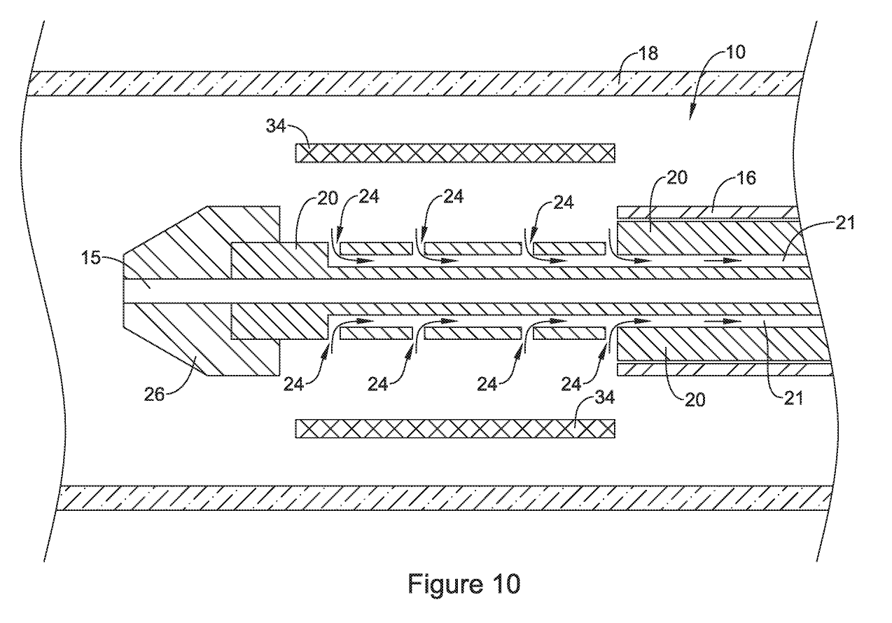

FIG. 10 shows stent 18 having been pulled radially inward away from the inner surface of lumen 18. Additionally, FIG. 10 shows arrows continuing to pull vacuum through ports 24 and delivery lumens 21. It can be appreciated that the vacuum may be applied by a clinician or operator with a device located external to the patient (e.g., a vacuum device attached to the proximal end of inner member 20). The vacuum may be applied continuously to draw stent 34 away from the inner surface of lumen 18 to a position in which stent 34 is disposed along stent receiving region 22. It can be appreciated that after stent 34 is pulled down onto stent receiving region 22 (as shown in FIG. 10), stent 34 may be repositioned within lumen 18 and then redeployed according to the methodology as described with respect to FIGS. 1-6.

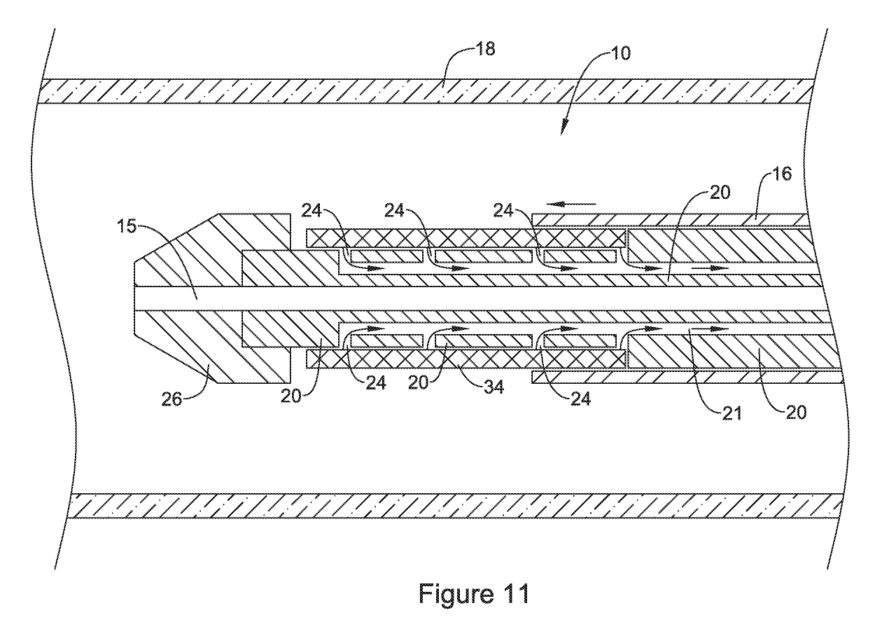

FIG. 11 shows stent 34 having been pulled radially inward and along stent receiving region 22 (shown in FIG. 9). Further, FIG. 11 shows retraction sheath 16 being extended in a distal direction such that sheath 16 extends over a portion of stent 34 to surround stent 34. In other words, FIG. 11 may illustrate the “recapturing” of stent 34 within the retraction sheath 16 of stent system 10.

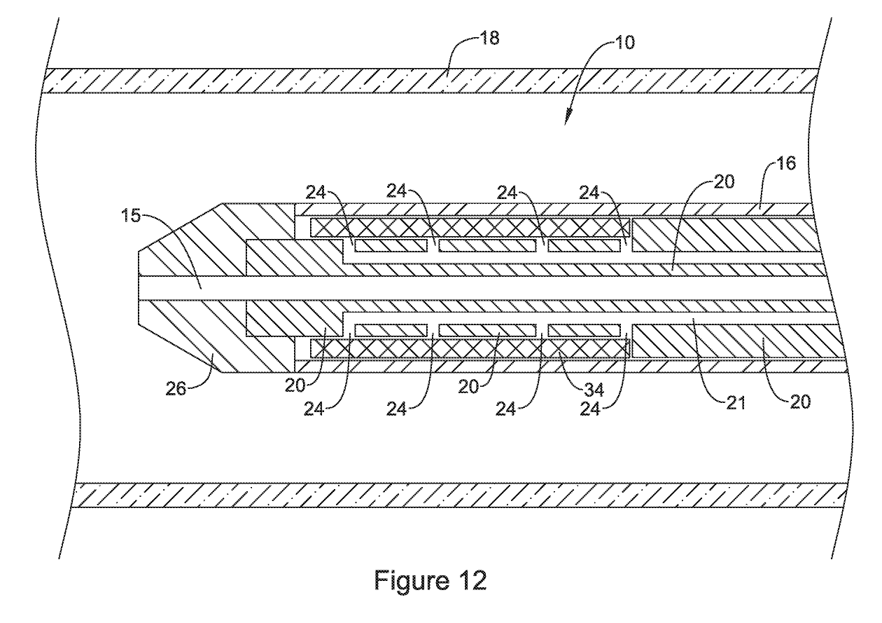

FIG. 12 shows stent 34 fully captured underneath stent retraction sheath 16. It can be appreciated that stent system 10 may be navigated to a different position within the body lumen and/or the patient's body and redeployed according any of the methods described herein. Alternatively stent system 10 may be used to remove stent 34 from the body lumen and/or the patient's body

The materials that can be used for the various components of system 10 (and/or other systems disclosed herein) may include those commonly associated with medical devices. For simplicity purposes, the following discussion makes reference to deployment sheath 16, and inner member 20. However, this is not intended to limit the disclosure as the discussion may be applied to other similar members and/or components of members or systems disclosed herein.

Deployment sheath 16, and inner member 20, and/or other components of system 10 may be made from a metal, metal alloy, polymer (some examples of which are disclosed below), a metal-polymer composite, combinations thereof, and the like, or any other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to above, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2-0.44% strain before plastically deforming.

In some embodiments, the linear elastic and/or non-super-elastic nickel-titanium alloy is an alloy that does not show any martensite/austenite phase changes that are detectable by DSC and DMTA analysis over a large temperature range. For example, in some embodiments, there may be no martensite/austenite phase changes detectable by DSC and DMTA analysis in the range of about −60° C. to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties and has essentially no yield point.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of deployment sheath 16 and inner member 20 may also be doped with, made of, or otherwise include a radiopaque material including those listed herein or other suitable radiopaque materials.

In some embodiments, a degree of MRI compatibility is imparted into system 10. For example, to enhance compatibility with Magnetic Resonance Imaging (MRI) machines, it may be desirable to make deployment sheath 16 and inner member 20, in a manner that would impart a degree of MRI compatibility. For example, deployment sheath 16 and inner member 20, or portions thereof, may be made of a material that does not substantially distort the image and create substantial artifacts (artifacts are gaps in the image). Certain ferromagnetic materials, for example, may not be suitable because they may create artifacts in an MRI image. Deployment sheath 16 and inner member 20, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Some examples of suitable polymers that may be used to form deployment sheath 16 and inner member 20, and/or other components of system 10 may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6% LCP.

In some embodiments, the exterior surface of the system 10 may include a coating, for example a lubricious, a hydrophilic, a protective, or other type of coating. Hydrophobic coatings such as fluoropolymers provide a dry lubricity which improves device handling and exchanges. Lubricious coatings improve steerability and improve lesion crossing capability. Suitable lubricious polymers may include silicone and the like, polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, the entire disclosures of which are incorporated herein by reference.

It should be understood that this disclosure is, in many respects, only illustrative. Changes may be made in details, particularly in matters of shape, size, and arrangement of steps without exceeding the scope of the disclosure. The disclosure's scope is, of course, defined in the language in which the appended claims are expressed.

What is claimed is:

1. A stent delivery system, comprising:
an inner shaft having a proximal portion, a distal portion and at least one lumen extending therein, the inner shaft further including a stent receiving region disposed along the distal portion;
a stent disposed along the stent receiving region;
wherein the inner member includes a plurality of apertures disposed along at least a portion of the stent receiving region;
wherein the plurality of apertures are configured to permit fluid to flow therethrough against an inner surface of the stent to assist expansion of the stent.

2. The stent delivery system of claim 1, further comprising a deployment sheath disposed about the inner shaft.

3. The stent delivery system of claim 2, wherein the stent is disposed between the deployment sheath and the inner shaft.

4. The stent delivery system of claim 1, wherein each of the plurality of apertures is in fluid communication with the at least one lumen of the inner shaft.

5. The stent delivery system of claim 4, wherein the at least one lumen extending within the inner shaft is configured to permit a guidewire to extend therethrough.

6. The stent delivery system of claim 1, wherein the inner shaft includes a plurality of lumens extending therein, and wherein at least one of the plurality of lumens is in fluid communication with each of the plurality of apertures.

7. The stent delivery system of claim 1, wherein each of the plurality of apertures are spaced apart from each other along the stent receiving region.

8. The stent delivery system of claim 1, further comprising a seal disposed in the at least one lumen distal of the plurality of apertures.

9. The stent delivery system of claim 1, wherein the plurality of apertures are configured to deliver fluid toward the inner surface of the stent such that the fluid expands the stent from a first position in which the stent is partially deployed to a second position in which the stent is fully deployed.

10. The stent delivery system of claim 1, wherein the plurality of apertures are further configured to withdraw fluid therethrough in order to create a vacuum sufficient to radially collapse the stent radially inward after being expanded.

11. A stent delivery system, comprising:
an inner shaft having a proximal portion, a distal portion and at least one lumen extending therein;
a deployment sheath positioned over the inner shaft;
a stent positioned between the inner shaft and the deployment sheath;
wherein the inner member includes a plurality of openings disposed along the distal portion;
wherein the plurality of openings are configured to permit fluid to flow therethrough to expand the stent from a first partially deployed position to a second fully deployed position.

12. The stent delivery system of claim 11, wherein the inner member further includes a stent receiving region positioned along the distal portion of the inner member.

13. The stent delivery system of claim 12, wherein the plurality of openings are located along the stent receiving region.

14. The stent delivery system of claim 13, wherein the stent is positioned along the stent receiving region such that the plurality of openings are directed at an inner surface of the stent.

15. The stent delivery system of claim 14, wherein the plurality of openings are in fluid communication with the at least one lumen.

16. The stent delivery system of claim 15, wherein each of the plurality of openings are spaced apart from each other along the stent receiving region.

17. The stent delivery system of claim 16, wherein each of the plurality of openings are designed to channel fluid radially away from a longitudinal axis of the inner member.

18. The stent delivery system of claim 17, wherein the plurality of openings are further configured to withdraw fluid therethrough in order to create a vacuum sufficient to radially collapse the stent radially inward after being expanded.

19. The stent delivery system of claim 11, wherein the inner shaft includes a plurality of lumens extending therein, and wherein at least one of the plurality of lumens is in fluid communication with each of the plurality of openings.

20. A method of deploying a stent, the method comprising:
advancing a stent delivery system to a target site within a patient, the stent delivery system comprising:
an inner shaft having a proximal portion, a distal portion and at least one lumen extending therein, the inner shaft further including a stent receiving region disposed along the distal portion;
a stent disposed along the stent receiving region;
wherein the inner member includes a plurality of apertures disposed along at least a portion of the stent receiving region, wherein the plurality of apertures are configured to permit fluid to flow therethrough;
expelling fluid through the plurality of apertures such that the fluid expands the stent from a first partially deployed position to a second fully deployed position.
